(19) United States
(12) Patent Application Publication
(10) Pub. No.: US 2017/0014264 A1
(43) Pub. Date: Jan. 19, 2017

(54) COLD PACK FOR BANDAGE

(71) Applicant: MURECK SISTERS, LLC, Las Vegas, NV (US)

(72) Inventors: Melissa Anne Bradley, Huntington Beach, CA (US); Andrew Michael Moeck, Huntington Beach, CA (US)

(21) Appl. No.: 15/206,050

(22) Filed: Jul. 8, 2016

Related U.S. Application Data
(60) Provisional application No. 62/192,471, filed on Jul. 14, 2015.

(51) Int. Cl. A61F 7/02; A61F 13/02; A61N 5/06; A61F 7/03; A61F 7/10

(52) U.S. Cl. CPC A61F 7/02; A61F 7/03; A61F 7/106; A61N 5/0616; A61F 13/0206; A61F 2007/0219; A61F 2007/108; A61N 2005/0626; A61N 2005/0661

(57) ABSTRACT

A bandage, such as an adhesive bandage or non-adhesive bandage, can be modified to include a cold pack. The cold pack may be placed in a pocket formed by a gauze sponge of the bandage and a backing of the bandage. The cold pack may also be adhered to a surface of the bandage. The cold temperature of the cold pack can, in some instances, reduce swelling at the wound site, possibly facilitate faster healing, and possibly provide psychological benefits that promote healing.

### INCORPORATION BY REFERENCE TO ANY RELATED APPLICATIONS

Any and all applications, if any, for which a foreign or domestic priority claim is identified in the Application Data Sheet of the present application are hereby incorporated by reference under 37 CFR 1.57.

### BRIEF DESCRIPTION OF THE DRAWINGS

Throughout the drawings, reference numbers may be re-used to indicate correspondence between referenced elements. The drawings are provided to illustrate embodiments of the inventions described herein and not to limit the scope thereof.

FIG. 1 depicts an example bandage.

FIG. 2 depicts an exploded view of an example bandage.

FIG. 3 depicts an embodiment of a bandage usage process.

FIG. 4 depicts another exploded perspective view of an example bandage.

### DETAILED DESCRIPTION

The disclosure provided in the following pages describes various example embodiments. The designs, figures, and description are non-limiting examples of some embodiments of the inventions described herein. Other embodiments may or may not include the features disclosed herein. Moreover, disclosed advantages and benefits may apply to only some embodiments and should not be used to limit the scope of the inventions described herein.

Bandages are commonly applied to wounds to stop bleeding and facilitate the healing process. Many bandages, including some adhesive bandages such as Band-Aid™ brand bandages, include adhesive wing portions and a central gauze sponge. The gauze sponge has absorbent properties and is typically placed directly on a wound to stanch blood flow. The adhesive wing portions are typically placed on an area of the skin other than the wounded area so as to hold the gauze sponge in place over the wound. Other types of bandages do not include an integral adhesive, but instead may be formed by applying a separate gauze sponge to the wound and then taping or otherwise securing the gauze sponge to the skin.

Both types of bandages, including bandages with integral adhesives and those without integral adhesives can be modified to include a cold pack. The cold temperature of the cold pack can, in some instances, reduce swelling at the wound site. As a result, the cold pack can reduce pain at the wound site and possibly facilitate faster healing. One possible additional benefit of including a cold pack with a bandage may be psychological. Children, for instance, may have greater confidence in the healing ability of a bandage with a cold pack. Thus, cold-pack bandages may provide greater comfort to children (and others) than existing bandages.

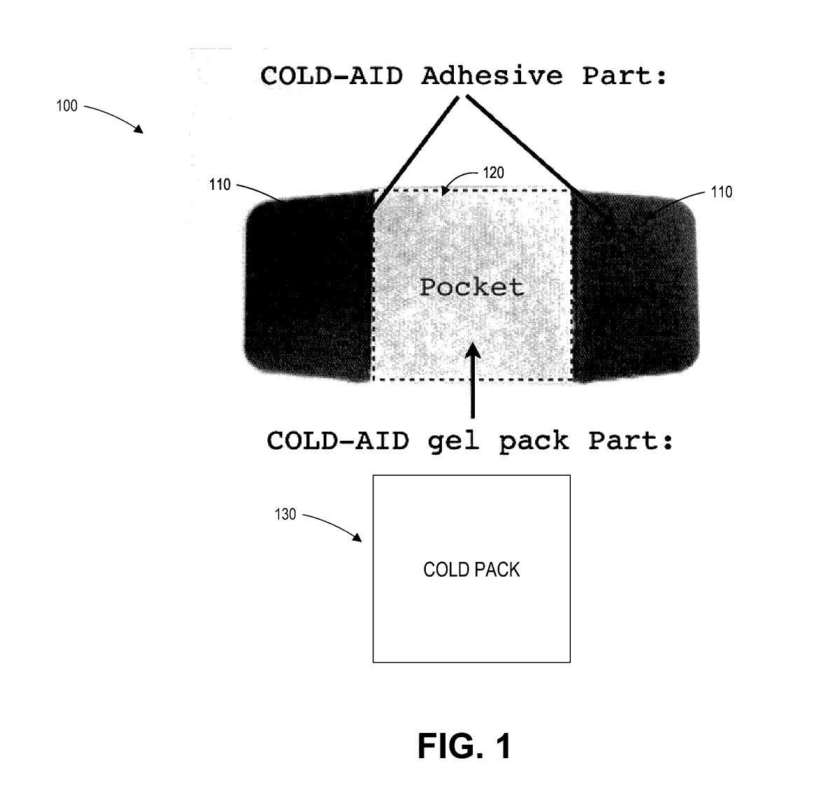

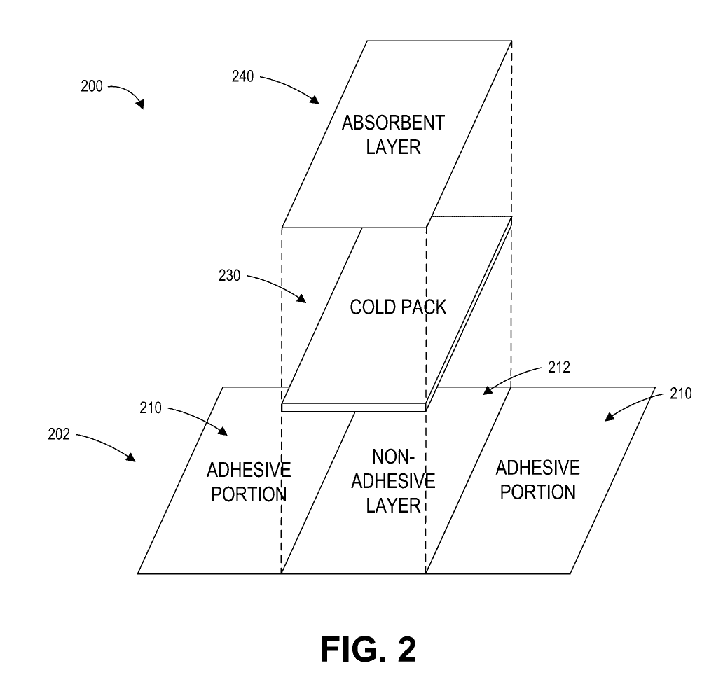

Referring to FIG. 1, an example bandage 100 is shown with an insertable cold pack 130. The bandage 100 is a Band-Aid™ style bandage having adhesive wings 110 and a gauze sponge 120. The gauze sponge 120 may be partially attached to the adhesive wings 110 so as to form a pocket with the bandage material behind it (see, e.g., FIG. 2, described below). The cold pack 130 can be inserted into this pocket.

When affixed to a person, the gauze sponge 120 can be placed in contact with a wound surface, and the cold pack 130 behind the gauze sponge 120 can apply a cold temperature through the gauze sponge 120 to the wound surface (effectively absorbing heat from the wound surface). The adhesive wings 110 can hold the bandage 110 and associated cold pack 130 in place against the skin of the person.

The cold pack 130 can be made of a plastic (or other material) enclosure that encloses a gel material or the like. In an embodiment, the cold pack 130 can be placed in a freezer or refrigerator (or other cold place) to attain a temperature colder than room temperature. The cold pack 130 may be removed from the freezer and inserted into the pocket of the bandage 100 prior to application of the bandage to the wound site. When the bandage 100 is later removed from the wound site, the cold pack 130 can be removed from the pocket and placed back in the freezer for subsequent use. Thus, cold packs 130 may be sold separately and/or together with bandages 110.

In other embodiments, the cold pack 130 can be an instant (or rapid) cold pack or the like that includes one or more chemicals that may create a cold temperature when the cold pack 130 is squeezed, snapped, twisted, or the like. Thus, the cold pack 130 need not be stored in a freezer or refrigerator in order to become cold in some embodiments. For instance, the cold pack 130 may include two bags; one containing water, which may be inside a bag containing a solid such as ammonium nitrate, calcium ammonium nitrate, or urea. When the inner bag of water is broken by squeezing the package, it is allowed to dissolve the solid in an endothermic reaction. This reaction absorbs heat from the surroundings, quickly lowering the cold pack's temperature.

Turning to FIG. 2, an exploded perspective view of an example bandage 200 is shown. The bandage 200 can include some or all of the features of the bandage 100 described above. In the depicted embodiment, the example bandage 200 includes a bandage substrate 202 that includes two adhesive wing portions 210 and a non-adhesive layer 212. An absorbent layer 240, which may include gauze or the like, can form a pocket with the non-adhesive layer 212. Both the non-adhesive layer 212 and the absorbent layer 240 do not include an adhesive in an embodiment so as to facilitate slipping a cold pack 230 in and out of the pocket. The absorbent layer 240 may be attached to the substrate 202 of the bandage 200 at two or three edges of the absorbent layer 240.

It should be noted that in other embodiments, the entire substrate 202 of the bandage 200 may include adhesive, such that the cold pack 230 is adhered to the substrate 202. Similarly, the back of the absorbent layer 240 may include adhesive that adheres to the cold pack 230 instead of (or in addition to) the substrate 202. Thus, in some embodiments the cold pack 230 is not a separate piece from the rest of the bandage 200, but rather is connected to other components of the bandage 200 and may thus be sold as a single unit with the bandage 200. The entire bandage 200 may therefore be stored in a freezer or refrigerator (or other cold place), or the cold pack 230 may be an instant cold pack as described above.

Although not shown, a cold pack may be coupled or packaged solely with a gauze sponge so that the cold pack may be used with bandages that do not have integral adhesives. Further, any of the bandages described herein can be of any size suitable to treat a given wound, including small bandages suitable for treating wounds on the face, knuckle bandages, larger bandages, and the like. Moreover, the bandages described herein are not limited to a rectangular shape, but instead may be circular, oval, ovaloid, square, triangle shaped, H-shaped (e.g., as in a knuckle bandage), or the like. In other embodiments, the cold pack is disposed in a pocket formed on the outside of the bandage, rather than a pocket formed with the absorbent layer 240. For instance, a pocket may be formed between the back of the non-adhesive layer 212 (which may actually have an adhesive in this embodiment to hold the absorbent layer 240 in place) and another surface. Similarly, the cold pack can be attached to the back of the layer 212 with an adhesive another embodiment. In yet another embodiment, the cold pack can be attached to the back of the layer 212 with a hook-and-loop fastener, such as a Velcro™ fastener or the like.

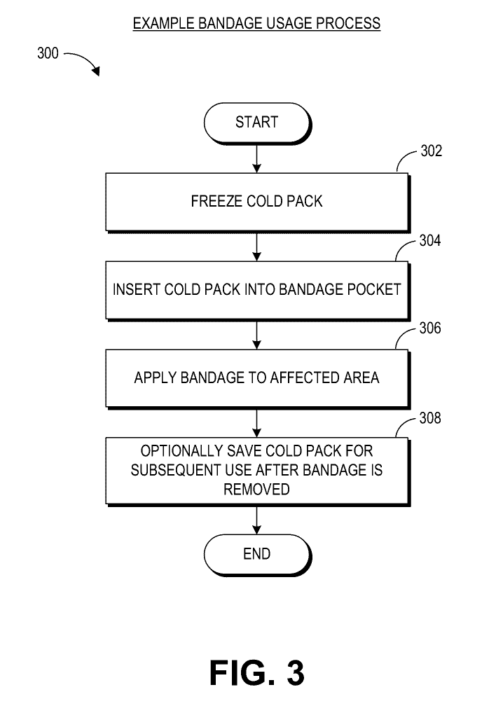

FIG. 3 depicts an embodiment of a bandage usage process 300. The bandage usage process 300 can be performed using any of the example bandages described herein.

At block 302, a cold pack is frozen, for example, by a user. The cold pack may be cooled rather than frozen in other embodiments. At block 304, once a person has need to use a bandage, the cold pack can be inserted into the bandage pocket. The bandage may then be applied to the effective area at block 306. The cold pack can optionally be saved for subsequent use after the bandage is removed at block 308.

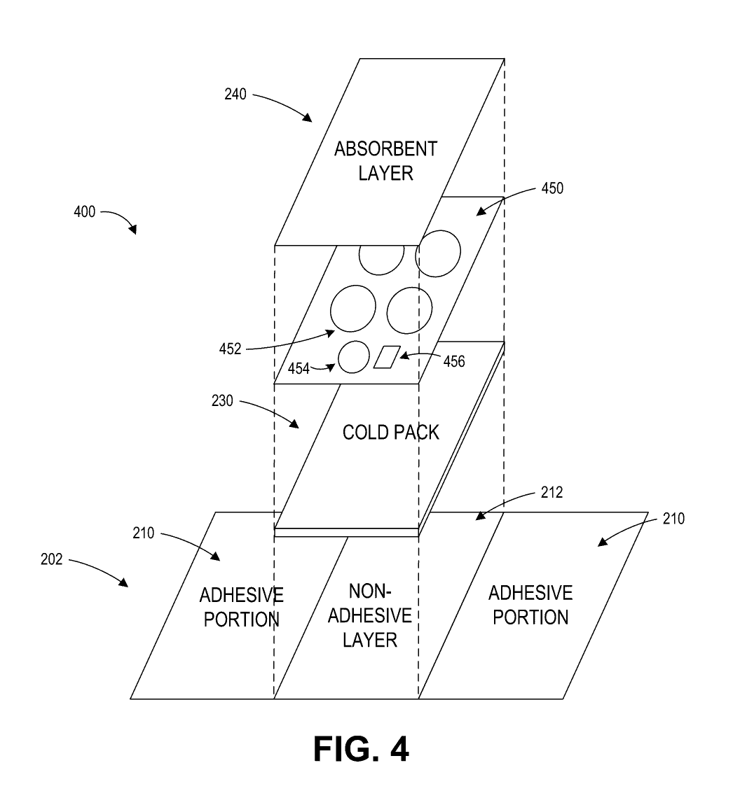

FIG. 4 depicts another exploded perspective view of an example bandage 400. The bandage 400 can include all the features of the bandages 100, 200 described above. In addition, the example bandage 400 shown includes a circuit layer 450, which includes light sources 452, a battery 454, and a controller or processor 456. The light sources 452 can be ultraviolet (UV) light sources, such as UV light-emitting diodes (LED) or the like. In some patients, UV light can help control or reduce infection. In addition, UV light can control or reduce itching from bug bites, stings, and the like.

The battery 454 can power the light sources 454, and the controller or processor 458 can control a duty cycle of the light sources 454. The controller or processor 458 may, for instance, include firmware, digital logic, and/or analog hardware configured to cause the light sources 454 to emit light at regular intervals, such as 10 seconds every hour that the bandage 400 is applied to a person, or a few seconds every 5-10 minutes, or the like. The controller or processor 458 may cause the light sources 454 to emit light in short bursts or in longer bursts.

While four light sources 452 are shown, there may be more or fewer in other embodiments, including one or more light sources 452. In an embodiment, the light sources 452 include one or more strips of UV LEDs. Although UV light may be used, the light sources 452 may also implement other wavelengths of light, including visible and infrared wavelengths. Further, the light sources 452 may emit light at different wavelengths.

The cold pack 230 may also be omitted in some embodiments, such that the bandage 400 includes the circuit layer 450 (with light sources 454) but not the cold pack 230. Thus, the light sources 454 may be coupled between the absorbent layer 240 and the non-adhesive layer 212 (which may be adhesive in an embodiment to adhere to the circuit layer 240).

### Terminology

Conditional language used herein, such as, among others, “can,” “could,” “might,” “may,” “e.g.,” and from the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

While the above detailed description has shown, described, and pointed out novel features as applied to various embodiments, it will be understood that various omissions, substitutions, and changes in the form and details of the logical blocks, modules, and processes illustrated may be made without departing from the spirit of the disclosure. As will be recognized, certain embodiments of the inventions described herein may be embodied within a form that does not provide all of the features and benefits set forth herein, as some features may be used or practiced separately from others.

What is claimed is:

1. A bandage comprising:
an adhesive surface configured to be adhered to skin of a person;
a pocket coupled with the adhesive surface, the pocket formed at least in part by an absorbent material configured to be placed on a wound of the person; and
a cold pack sized so as to be inserted into the pocket.

2. The bandage of claim 1, wherein the cold pack comprises a gel material.

3. The bandage of claim 1, wherein the cold pack is configured to be placed in a freezer, refrigerator, or the like.

4. The bandage of claim 1, wherein the cold pack is configured to be inserted into the pocket after being cooled to a temperature lower than room temperature.

5. The bandage of claim 1, wherein the cold pack comprises one or more chemicals configured to produce an endothermic reaction in response to being disturbed.

6. The bandage of claim 1, wherein the absorbent material comprises gauze.

7. A bandage comprising:
an adhesive portion configured to be adhered to skin of a person;
a cold pack coupled with the adhesive portion; and
an absorbent material coupled with the cold pack and configured to be placed on a wound of the person.

8. The bandage of claim 7, wherein the cold pack is adhered to the adhesive portion, and wherein the absorbent material is adhered to the cold pack.

9. The bandage of claim 7, wherein the absorbent material comprises gauze.

10. The bandage of claim 7, wherein the cold pack is coupled with the adhesive portion through a pocket formed between a non-adhesive surface of the adhesive portion and the absorbent material.

11. A bandage comprising:
an adhesive portion configured to be adhered to skin of a person;
an absorbent material configured to be placed on a wound of the person; and
a light source configured to shine light on the wound of the person through the absorbent material.

12. The bandage of claim 11, wherein the light source comprises an ultraviolet (UV) light source.

13. The bandage of claim 11, further comprising a battery.

14. The bandage of claim 11, further comprising a processor configured to control the light source.

15. The bandage of claim 14, wherein the processor is configured to control a duty cycle of light emission from the light source.

16. The bandage of claim 11, wherein the light source comprises a plurality of light sources.

17. The bandage of claim 11, further comprising a cold pack.
